(19) United States
(12) Patent Application Publication
(10) Pub. No.: US 2005/0220888 A1
(43) Pub. Date: Oct. 6, 2005

(54) Controlled release compositions and methods for using same

(76) Inventors: Lakahmi Putcha, Houston, TX (US); Joe McDonough, Helotes, TX (US); Edward J. Boland, San Antonio, TX (US); Hong Dixon, Helotes, TX (US); Joseph T. Persyn, Lakehills, TX (US); Niraj Vasishtha, San Antonio, TX (US)

Correspondence Address: PAULA D. MORRIS;THE MORRIS LAW FIRM, P.C., 10260 WESTHEIMER, SUITE 360, HOUSTON, TX, 77042-3110, US

(21) Appl. No.: 10/499,996

(22) PCT Filed: Jan. 31, 2003
(86) PCT No.: PCT/US03/02797

§ 371 Date: Jun. 22, 2004

Related U.S. Application Data
(60) Provisional application No. 60/353,633, filed on Jan. 31, 2002.
(60) Provisional application No. 60/353,766, filed on Jan. 31, 2002.

(51) Int. Cl. A61K 9/22; A61K 9/16; A61K 9/50

(52) U.S. Cl. 424/490

(57) ABSTRACT

Pharmaceutical preparations adapted for mucosal delivery, preferably for nasal delivery, which can be easily and safely used over days to weeks with minimal side effects. The pharmaceutical preparations comprise microcapsules comprising at least one pharmaceutically active agent. The microcapsules provide controlled release of the pharmaceutically active agent. Cytotoxicity is avoided for cytotoxic pharmaceutically active agents and/or for cytotoxic dosages by one or more of the following: (a) manipulating the mucosal transport rate of the pharmaceutically active agent through the mucosal bodies to achieve a transport rate which is substantially the same as the controlled release rate, and/or (b) selecting only a most active and/or less cytotoxic enantiomer of the pharmaceutically active agent for use in the pharmaceutical preparation.

The present application claims the benefit of U.S. Provisional Application Ser. No. 60/353,766 and U.S. Provisional Application Ser. No. 60/353,633, both filed Jan. 31, 2002.

### GOVERNMENT RIGHTS

The U.S. government has certain rights in this invention pursuant to grant number NAG 9-1300 from the National Aeronautics and Space Administration.

### FIELD OF THE INVENTION

The present application relates to the field of pharmacology and medicinal chemistry, and provides improved pharmaceuticals, and methods for effective administration thereof.

### BACKGROUND OF THE INVENTION

Allergies often are chronic in nature. Medication that controllably releases over a long period of time would be most effective for the control of allergies. However, allergies typically are treated with injections, pills, or capsules, which do not provide controlled release of the allergy medication.

Motion sickness occurs in humans when they are exposed to unfamiliar movement or visual stimulus. The characteristic symptoms are nausea and vomiting that disrupt normal function until these symptoms ameliorate. Astronauts frequently experience space motion sickness and disorientation as a result of changes in gravitational level. This results in a loss of work time and a disruption of planned activities until symptoms are relieved, often resulting in a loss of expensive flight programs and experiments.

Effective pharmaceutical preparations are needed to treat motion sickness, allergies, and a wide variety of ailments, which can be easily and safely used over days to weeks with minimal side effects.

### SUMMARY OF THE INVENTION

The present application provides a pharmaceutical preparation adapted for mucosal delivery of a pharmacologically effective dose of a pharmacologically active agent to a mammal. The pharmaceutical preparation comprises microcapsules adapted to provide controlled release of the pharmacologically effective dose. The microcapsules comprise a core and a shell, the shell comprising a release retardant, the core comprising the pharmacologically active agent and an excipient. The pharmacologically active agent is selected from the group consisting of antihistamines and anticholinergics.

In another aspect, the application provides a pharmaceutical preparation adapted for mucosal delivery of a pharmacologically effective dose of a pharmacologically active agent to a mammal. The pharmaceutical preparation comprises microcapsules adapted to provide controlled release of the pharmacologically effective dose. The microcapsules comprise a shell and a core, the core comprising a quantity of a single enantiomer of the pharmacologically active agent. The pharmacologically active agent is selected from the group consisting of antihistamines and anticholinergics.

In another aspect, the application provides a pharmaceutical preparation adapted for mucosal delivery of a pharmacologically effective dose of a pharmacologically active agent to a mammal. The pharmaceutical preparation comprises one or more absorption enhancers and microcapsules adapted to provide controlled release of the pharmacologically effective dose of the pharmacologically active agent. The pharmacologically active agent is selected from the group consisting of antihistamines and anticholinergics.

The application also provides a method for mucosal delivery of a pharmacologically effective dose of a pharmacologically active agent to a mammal. The method comprises:
providing a pharmaceutical preparation comprising microcapsules comprising a core and a shell, the shell comprising a release retardant, the core comprising a pharmacologically active agent and an excipient, wherein the pharmacologically active agent is selected from the group consisting of antihistamines and anticholinergics; and,
mucosally administering the pharmaceutical preparation to the mammal.

In yet another aspect, the application provides a method for mucosal delivery of a pharmacologically effective dose of a pharmacologically active agent to a mammal. The method comprises:
providing a pharmaceutical preparation comprising microcapsules adapted to provide controlled release of said pharmacologically effective dose, the microcapsules comprising a shell and a core, the core comprising a quantity of a single enantiomer of the pharmacologically active agent, wherein the pharmacologically active agent is selected from the group consisting of antihistamines and anticholinergics; and
mucosally administering the pharmaceutical preparation to the mammal.

In another aspect, the application provides a method for mucosal delivery of a pharmacologically effective dose of a pharmacologically active agent to a mammal. The method comprises:
providing a pharmaceutical preparation comprising one or more absorption enhancers and microcapsules adapted to provide controlled release of the pharmacologically effective dose of the pharmacologically active agent, wherein the pharmacologically active agent is selected from the group consisting of antihistamines and anticholinergics.
mucosally administering the pharmaceutical preparation to the mammal.

In yet another aspect, the application provides a pharmaceutical preparation for mucosal delivery of a pharmacologically active agent to a mammal without cytotoxicity to mucosal epithelial cells. The pharmaceutical preparation comprises:
microcapsules comprising a shell and a core comprising a quantity of one or more pharmacologically active agents selected from the group consisting of antihistamines and anticholinergics, the microcapsules being adapted to release the one or more pharmacologically active agents at a release rate, wherein cytoxicity is predicted due to a factor selected from the group consisting of the release rate and inherent cytotoxicity of the pharmacologically active agent; and
one or more absorption enhancers effective to produce a mucosal transport rate which is substantially the same as the release rate of said pharmacologically active agent, thereby preventing cytotoxicity.

In another embodiment, the application provides a method for mucosal delivery of a pharmacologically active agent to a mammal. The method comprises:
providing a pharmaceutical preparation comprising microcapsules comprising a shell and a core, the core comprising one or more pharmacologically active agents selected from the group consisting of antihistamines and anticholinergics, the microcapsules being adapted to provide a release rate of the pharmacologically active agent, wherein cytotoxicity is predicted due to a factor selected from the group consisting of the release rate and inherent cytotoxicity of the pharmacologically active agent; and,
mucosally delivering the pharmaceutical preparation to a mammal under conditions effective to produce a mucosal transport rate which is substantially the same as the release rate of the pharmacologically active agent, thereby preventing cytotoxicity.

In another aspect, the application provides a method for alleviating a condition in a mammal selected from the group consisting of motion sickness, allergy, and a combination thereof. The method comprises administering to the mammal a pharmacologically effective amount of a highest pharmacological activity enantiomer of a phenothiazine.

In another aspect, the application provides for resolving (+) enantiomer and (−) enantiomer of ethopropazine, said method comprising:
purifying a racemic ethopropazine free base;
mixing the racemic ethopropazine free base solution and an optically active organic acid under mixing conditions effective to produce a precipitate comprising crystals comprising diasteriomers comprising a reaction product between the optically active organic acid and a corresponding enantiomer of the ethopropathiazine; and,
recrystallizing at least one of the diasteriomers.

### BRIEF DESCRIPTION OF THE DRAWINGS

FIG. 1 depicts the X-ray diffraction spectrum of promethazine (PMZ) racemate.

FIG. 2 depicts the X-ray diffraction spectrum of the (+) enantiomer of PMZ.

FIG. 3 depicts the X-ray diffraction spectrum of the (−) enantiomer of PMZ.

FIG. 4 depicts the electrophoretic separation of IL-6 amplification products resulting from treatment of HUVEC cells with histamine, racemic PMZ, and the (+)- and (−) enantiomer of PMZ. The top portion of the figure is IL-6, the bottom HPRT (control gene).

FIG. 5 depicts the IL-6 production by HUVEC Cells exposed to histamine, the racemate, (+), and (−) enantiomers of PMZ at 10⁻⁵ molar. The measurements reflected in FIGS. 5-7 and 17 are of densitometry readings measured using the Kodak 1-D gel quantitation software package. The numbers have no units as these are eliminated by division during the data calculation.

FIG. 6 depicts the IL-6 production by HUVEC Cells exposed to histamine, the racemate, (+), and (−) enantiomers of ethopropazine (EPZ) at 10⁻⁵ molar.

FIG. 7 depicts the IL-6 production by HUVEC Cells exposed to histamine, the racemate, (+), and (−) enantiomers of trimeprazine (TPZ) at 10⁻⁵ molar.

FIG. 8 is a picture of the microcapsules produced in Example 8.

FIG. 9 is a plot of % cell survival from the cytotoxicity testing of the (+) enantiomer of promethazine for one hour, from Example 3.

FIG. 10 is a plot of the % cell survival from the cytotoxicity testing of the (−) enantiomer of promethazine for one hour, from Example 3.

FIG. 11 is a plot of the % cell survival from the cytotoxicity testing of the racemate of promethazine for one hour, from Example 3.

FIG. 12 illustrates the histology of the saline formulation of Example 10.

FIG. 13 illustrates the histology of the PMZ in saline formulation of Example 10.

FIG. 14 illustrates the histology of the PMZ-PBS formulation of Example 10.

FIG. 15 illustrates the histology of the PMZ-Freebase formulation of Example 10.

FIG. 16 illustrates the histology of the encapsulated PMZ formulation in PEG-Glycofurol of Example 10.

FIG. 17 depicts the IL-6 production by HUVEC Cells exposed to histamine, the racemate, the (−)-enantiomer of EPZ (#1), and the (+)-enantiomer of EPZ (#2) at 10⁻⁶ molar.

### DETAILED DESCRIPTION

The present application provides pharmaceutical preparations adapted for mucosal delivery which can be easily and safely used over days to weeks with minimal side effects. A preferred type of mucosal delivery is nasal delivery.

The pharmaceutical preparations comprise microcapsules comprising at least one pharmacologically active agent selected from the group consisting of antihistamines and anticholinergics. The microcapsules provide controlled release of the pharmacologically active agent. Cytotoxicity is avoided for cytotoxic pharmacologically active agents and/or for cytotoxic release rates of the pharmacologically active agent by one or more of the following: (a) manipulating the mucosal transport rate of the pharmacologically active agent through the mucosal epithelial cells to achieve a mucosal transport rate which is substantially the same as the controlled release rate, and/or (b) selecting only a most active enantiomer, to allow less to be used, and/or a less cytotoxic enantiomer of the pharmacologically active agent for use in the pharmaceutical preparation.

Many organic compounds exist in optically active forms, i.e., they have the ability to rotate the plane of plane-polarized light. In describing an optically active compound, the prefixes D and L, R and S, or (+)- or (−)-, are used to denote the absolute configuration of the molecule about its chiral center(s). The enantiomers of a racemic drug generally differ in biological activity as a consequence of stereoselective interaction with optically active biological macromolecules. For drugs having a specific action at receptors, one enantiomer may have all of the activity, whereas the other enantiomer appears to be inactive. Such a molecule may be marketed by the pharmaceutical industry as a racemate, assuming that the non-active enantiomer is insignificant from a therapeutic and a toxicological point of view. However, the non-active enantiomer may actually be deleterious rather than simply inert and it is likely that the side-effects encountered may be due to the non-active enantiomer.

Many biological receptors are chirally sensitive, including the histamine receptors. Waelbroeck M, Camus J, Tastenoy M, et al. Stereoselective interaction of procyliine, hexahydrodifenidol, hexabutinol and oxyphencyclimine and of related antagonists, with four muscarinic receptors. Eur. J. Pharmacol. 227:3342, (1992), incorporated herein by reference. Different enantiomers of various chiral antagonists also show differing levels of inhibition. Hence, phenothiazine enantiomers, such as PMZ enantiomers, may have different affinities for the histamine receptors, resulting in different efficacies in vivo.

Where the enantiomers of the particular pharmacologically active agent have different affinities for the relevant receptors, or demonstrate different cytotoxicity levels, a preferred embodiment comprises the use of only the (+)- or the (−)-enantiomer of the pharmacologically active agent. Preferably, the enantiomer exhibiting increased affinity for the receptor and/or lower cytotoxicity, preferably both, is chosen as the pharmacologically active agent in formulating the pharmaceutical preparation and in performing the method described herein.

Controlled delivery may be desirable for many pharmacologically active agents. Hence, mucosal delivery of pharmaceutical preparations comprising microcapsules comprising the pharmacologically active agent(s) may be used for a number of pharmacologically active agents, including but not necessarily limited to those selected from the group consisting of antihistamines and anticholinergics.

Controlled delivery of the pharmacologically active agent involves encapsulating the pharmacologically active agent in microcapsules. The microcapsules preferably comprise a core comprising one or more pharmacologically active agents. In a preferred embodiment, the core comprises an excipient. The core also preferably comprises one or more mono-, di-, and/or triglycerides, more preferably stearine, even more preferably partially hydrogenated palm oil. A preferred partially hydrogenated palm oil is CAS 68514-74-9. The core of the microcapsules is coated by a shell material comprising a release retardant, more preferably ethylcellulose, most preferably ethylcellulose of premium grade from about 4 to about 10, preferably comprising an ethoxyl content of from about 45 wt. % to about 47 wt. %. In a preferred embodiment, a 5% solution of ethylcellulose in 80% toluene and 20% ethanol has a viscosity of from about 9 centipoise (cP) to about 11 cP at 25° C. In a most preferred embodiment, the pharmaceutical formulation comprises absorption enhancers effective to increase the rate of mucosal transport of the pharmacologically active agent across the mucosal epithelium, preferably to a mucosal transport rate that is substantially the same as the controlled release rate.

The pharmaceutical preparations and methods will be described with reference to agents which are pharmacologically active to treat motion sickness and/or allergy. However, the pharmaceutical preparations and methods of the present application are not limited to pharmaceutical preparations and methods for treating motion sickness and/or allergy. Rather, the pharmaceutical preparations are useful to treat a variety of ailments using a pharmacologically active agent selected from the group consisting of antihistamines and anticholinergics.

Referring to agents for treating motion sickness, the source of the motion sickness response is complex. Although the semicircular canals and otolith organs are essential for the genesis of motion sickness, subsequent events leading to motion sickness take place in the CNS. Emesis, the final event in motion sickness, is a reflex controlled by the brain stem. A variety of pharmacological agents are effective in minimizing motion-induced emesis therapeutically. These agents include, but are not necessarily limited to antihistamines and anticholinergics.

Promethazine (PMZ) is a member of a class of compounds called phenothiazines. PMZ acts as a histamine receptor 1 (H₁) antagonist. PMZ also is effective against allergy symptoms. PMZ commonly is used clinically to prevent the symptoms of motion sickness during space flight and sea voyaging because PMZ is capable of halting the nausea and disorientation after onset. The H₁ receptor antagonism activity of PMZ is the apparent mechanism of action for the reduction of the symptoms of motion sickness. Interestingly, PMZ is a chiral compound that is used clinically as the racemate.

Promethazine (PMZ) has been isolated, resolved, and tested for cytotoxicity. Neither enantiomer of PMZ demonstrated a significant increase in cytotoxicity compared to the racemate. However, both of the enantiomers and the racemate of PMZ showed a significant level (10 ⁻⁴ molar) of inherent cytotoxicity. The (+)-enantiomer (as measured in water) of promethazine (PMZ) has been found to be the highest activity enantiomer of the racemic PMZ mixture. The (−) enantiomer (as measured in water) of ethopropazine has been found to be the highest activity enantiomer of the racemic ethopropazine mixture. As hereinafter used, the terms (+) and (−)-enantiomer refer to optical rotation as measured in water.

Useful compounds for administration to a patient include pharmaceutically acceptable acid addition salts of the pharmacologically active agent, preferably the phenothiazines defined by the above formula. Acids commonly employed to form such salts are inorganic acids, such as hydrochloric acid, hydrobromic acid, hydroiodic acid, sulfuric acid, phosphoric acid, and the like, and organic acids, such as p-toluenesulfonic, methanesulfonic acid, oxalic acid, p-bromophenylsulfonic acid, carbonic acid, succinic acid, citric acid, benzoic acid, acetic acid, and the like.

Examples of such pharmaceutically acceptable salts thus are the sulfate, pyrosulfate, bisulfate, sulfite, bisulfite, phosphate, monohydrogenphosphate, dihydrogenphosphate, metaphosphate, chloride, bromide, iodide, acetate, propionate, decanoate, caprylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, hydroxybutyrate, glycollate, tartrate, methanesulfonate, propanesulfonate, naphthalene-1-sulfonate, naphthalene-2-sulfonate, mandelate, and the like. Preferred pharmaceutically acceptable acid addition salts are those formed with mineral acids such as hydrochloric acid, hydrobromic acid and organic acids such as acetic acid, oxalic acid, maleic acid or fumaric acid.

It is important to note that the pharmacologically active agent preferably is not obtained from a commercially available tablet that may contain a variety of non-active ingredients, including binders, which may exert a detrimental effect on the efficacy of the composition. If the source of a pharmacologically active agent is a commercial tablet, then the mixture obtained from the tablet preferably is treated to provide the active ingredient relatively free, preferably substantially free of the non-active components. Methods of purification are well known to those of ordinary skill and may include dissolution of the mixture in a solvent and recrystallization, for example.

The pharmaceutical preparation may be used prophylactically, or may be administered to a patient already suffering from an ailment or symptoms associated therewith, such as allergy or motion sickness. Once relief has been provided, the composition can be administered under a regimen to maintain a substantially symptom-free state. Generally, the dosage or frequency of administration of the pharmacologically active agent required to keep the patient essentially free of allergy or motion sickness symptoms (the “maintenance dosage”) is less than the dosage or frequency used in the initial phase of treatment (the “initial dosage”) and lower than the dosages used with the racemate. After administration of the initial dosage, the dosage or frequency can be cut back until the symptoms begin to manifest themselves once again. The dosage or frequency is then adjusted to just suppress the symptoms.

As used herein, the term “phenothiazine” refers to compounds having the following general structure: 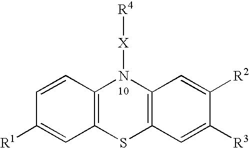
wherein
R¹, R², and R³ are limited primarily by size, preferably having a size substantially equivalent to an alkyl radical having 6 or fewer carbon atoms. In a preferred embodiment, R¹, R², and R³ independently are selected from the group consisting of hydrogen, a hydroxyl radical, an alkoxy radical comprising an alkyl radical having from about 1 to about 6 carbon atoms, an acyloxy radical comprising an alkyl radical having from about 1 to about 6 carbon atoms, a substituted or unsubstituted branched or unbranched alkyl radical having a total of from about 1 to about 6 carbon atoms, a substituted or an unsubstituted phenyl radical or a substituted or an unsubstituted benzyl radical wherein said substituted radicals comprise substituents selected from the group consisting of hydroxyl radicals, halogens, alkyl radicals having a total of from about 1 to about 6 carbon atoms, cyclic alkylene groups and heterocyclic alkylene groups having from about 4 to about 6 carbon atoms comprising a heterocyclic element selected from the group consisting of nitrogen or sulfur. In another embodiment, R¹, R², and R³ independently are selected from the group consisting of ionizable groups selected from the group consisting of ammonium, sulfonium, and phosphonium groups and esters thereof. The esters preferably comprise linear or branched alkyl groups comprising from about 1 to about 5 carbon atoms;
X is a linear or branched alkyl radical or an alkenyl group having from about 1 to about 5 carbon atoms;
R⁴ is a tertiary amine or thiol radical having the structure N—(R⁵)₃ or S—R⁵ wherein R⁵ may be the same or different entities independently selected from the group consisting of hydrogen, alkyl radicals and alkenyl radical or fluoroalkyl, having from about 1 to about 6 carbon atoms, preferably 1 to about 3 carbon atoms, cyclic alkylene groups and heterocyclic alkylene groups having from about 4 to about 6 carbon atoms comprising a heterocyclic element selected from the group consisting of nitrogen or sulfur.

Phenothiazines primarily differ by substitution of various alkylamino groups on the nitrogen atoms at the 10 position of the basic phenothiazine nucleus. The chemical group bound at the 10 position of the phenothiazine nucleus appears to determine histaminic response.

The method may use a racemic mixture, or only the (+)- or the (−)-enantiomer of a given pharmacologically active agent, such as a phenothiazine, to treat motion sickness, allergy, or other ailment. The following are the structures of certain preferred phenothiazines for use in the method: 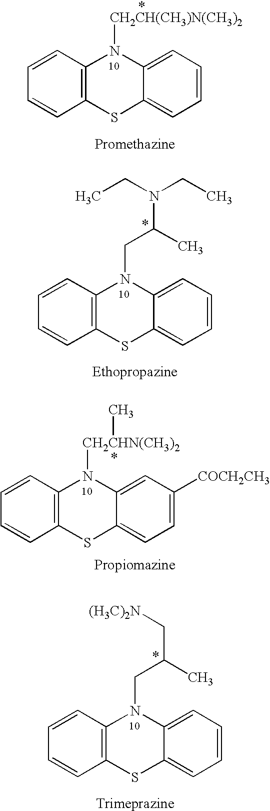
Promethazine, ethopropazine, and trimeprazine are available commercially as racemic mixtures, for example, from Aldrich Chemical Co., or by prescription.

Promethazine hydrochloride is currently administered during space flight after onset of motion sickness by a painful and unwieldy intramuscular route. A less invasive, more selective delivery route is preferred for safer, more effective remedies. Mucosal delivery, preferably nasal delivery, is noninvasive and should be amenable to space flight use. Importantly, nasal delivery also enables high plasma loadings without first pass metabolism in the liver after administration. This route is ideal for drugs, such as promethazine, that are rapidly metabolized to their inactive sulfoxide by liver oxidases.

In previous research, racemic promethazine hydrochloride was encapsulated in a variety of shell materials and administered to beagles; however, severe nasal irritation was observed. R. Ramanathan, R. S. Geary, L. Putcha, “Bioavailability of Intranasal Promethazine Dosage Forms in Dogs”, Pharmacol. Res. 38 (1), 1998, pg. 36-39, incorporated herein by reference.

Nasal delivery can be done by powder insufflation, aerosol delivery of droplets, liquid dosing or by application of a cream or ointment. Insufflation, aerosol, and liquid all have disadvantages such as microbiological instability, short residence time of dose, variable site of deposition, and variable dose. Supporting work has shown the importance of nasal ciliary beat frequency and site of deposition on the absorption of insulin. S. Gizurarson, E. Bechgaard, “Intranasal Administration of Insulin to Humans”, Diab. Res. Clin. Pract. 12, 1991, pg. 71-84, incorporated herein by reference. Site of administration of nasally delivered drugs also is important.

Recent developments in nasal administration of creams or gels by addition of absorption enhancers, such as polyethylene glycol 300 or 400 and dimethylcyclodextrin, have made this delivery mode highly desirable since problems of variable site deposition, dose and residence time are more manageable. E. Martin, N. G. M. Schipper, F. W. H. M. Merkus, “Nasal Mucociliary Clearance as a Factor in Nasal Drug Delivery”, Adv. Drug Deliv. Rev., 29, 1998, pg. 13-38; R. Ramanathan, R. S. Geary, L. Putcha, “Bioavailability of Intranasal Promethazine Dosage Forms in Dogs”, Pharmacol. Res. 38 (1), 1998, pg. 36-39, incorporated herein by reference.

Microencapsulation of the pharmacologically active agent, such as phenothiazine, achieves “controlled release” of the agent. In the case of phenothiazine, the release rate is effective to enable the composition to act as an “H1 receptor antagonist.” By “H1 receptor antagonist” is understood to mean that the phenothiazine is capable of partially or completely inhibiting the biological effect of histamine on the H1 receptor. An H1 receptor antagonist induces a coherent pharmacological response (including or not including its binding to the H1 receptor), specifically a reduced production of IL-6 in comparison to a control, in the assay described in Delneste Y., Lassalle P. et al Histamine induces IL-6 production by human endothelial cells. Clin. Exp. Immunol. 98:344-349, (1994), incorporated herein by reference. A preferred microcapsule composition comprises about 0.1 to 50% by weight of the phenothiazine, preferably about 20% by weight of the phenothiazine. Preferably, the release rate into isotonic saline at 37° C. takes 20-360 minutes.

Cytotoxicity has been avoided even when the pharmacologically active agent is inherently cytotoxic, or when the release rate is sufficient to cause cytotoxicity, by combining microencapsulation effective to achieve controlled release of the pharmacologically active agent with the use of absorption enhancers which transport the pharmacologically active agent through the cells at the site of administration, typically mucosal bodies, at a mucosal transport rate which is substantially the same as the controlled release rate. This combination of controlled release and rapid absorption caused by the absorption enhancers maintains the effective concentration in the cells at the site of administration below the cytotoxic limit. The absence of cytotoxicity symptoms using the foregoing combination has been demonstrated in the case of cytotoxic phenothiazines and nasal administration (see examples). It is believed that use of the same technique will avoid cytotoxicity using other cytotoxic agents selected from the group consisting of antihistamines and anticholinergics.

Enantiomer Resolution

Where one enantiomer of the pharmacologically active agent is more active and/or less cytotoxic, preferably both, it is preferred to use the more active, less cytotoxic enantiomer only in the pharmaceutical preparation. Methods of resolving enantiomers are known. For example, in order to resolve a phenothiazine racemate into its two enantiomers, 0.5-25 grams of optically pure phenothiazine enantiomers are isolated using column chromatography. Nilsson, J. Lars G.; Hermansson, Joeergen; Hacksell, Uli; Sundell, Staffan “Promethazine-resolution, absolute configuration and direct chromatographic separation of the enantiomers” Accta Pharm. Suec. (1984), 21 (5), 309-16, incorporated herein by reference. Generally, the racemate is allowed to react with an optically active compound. The two products of the reaction are diastereomers, which are separated by virtue of differences in their physical properties, such as solubility. The diastereomers are decomposed, and the optically active components of the original racemate are recovered. If the racemate is a base, an optically active acid or derivative thereof such as tartaric acid, or mandelic acid, is used to split the enantiomeric pair. In the case of phenothiazines, a preferred optically active acid is dibenzoyl tartaric acid. The racemate is mixed with the acid, and diastereomerically related and optically active salts crystallize. Since the diastereomeric salts have different solubility properties, they are separated by fractional crystallization to give homogeneous substances.

Alternately, the racemate may be separated using chromatographic separation, such as gas chromatography (GC), high performance liquid chromatography (HPLC) [Ponder, Garratt W.; Butram Sandra L.; Adams, Amanda G.; Ramanathan Chandra S.; Stewart, James T. “Resolution of promethazine, ethopropazine, trimeprazine and trimipramine enantiomers on selected chiral stationary phases using high-performance liquid chromatography,” Journal of Chromatography A, (1995), 692, 173-182, incorporated herein by reference], and recently capillary electrophoresis (CE) [Wang, Rongying; Lu Xiaoning; Wu, Mingjia “Chiral separation of promethazine by capillary electrophoresis with end-column amperometric detection” J. Sep. Sci. (2001), 24, 658-62, incorporated herein by reference]. In these chromatographic separations, a variety of chiral selectors have been employed, including proteins, modified crown ethers, and cyclodextrins.

Enantiomer Characterization

The enantiomers of ethopropazine (EPZ), trimeprazine (TPZ), and promethazine (PMZ) have been isolated and resolved, and the enantiomers of PMZ and ethopropazine have been tested for efficacy (as discussed above). Each enantiomer lot of phenothiazine is characterized to provide consistency within the test articles and to protect against varied polymorphism surprises. Once isolated, each drug class is characterized to determine its polymorph fingerprint vs. the racemate by powder diffraction x-ray (XRD). The XRD characterization is important because polymorphisim often occurs in chiral compounds. J. Breu, H. Domel, N. Per-Ola, Eur. J. Inorg. Chem. 11, 2000, pg. 2409-2419. H. H Paradies, S. F. Clancy, Rigaku J. 17 (2), 2000, pg. 20-35, incorporated herein by reference. A change in polymorph of a compound can result in a significant difference in the solubility and bioavailability of that compound. Chiral High Performance Liquid Chromatograpy (“Chiral HPLC”) was used along with optical rotation measured in water to determine the optical purity of the samples prepared. G. W. Ponder, S. L. Butram, A. G. Adams, J. T. Stewart, Resolution of Promethazine, Ethopropazine, Trimeprazine and Trimipramine Enantiomers on Chiral Stationary Phases Using HPLC, Jrnl. Chrom. A, 692, 1995, pg. 173-182, incorporated herein by reference. Nuclear Magnetic Resonance (NMR) and Infrared (IR) spectra and melting point information were gathered on each enantiomer. After complete characterization of each enantiomer, samples were set aside and retained as standards. Each subsequent lot of phenothiazine enantiomer prepared was analyzed against these primary standards prior to formulation and dosing.

The optical purity of the phenothiazine enantiomers was determined using Chiral HPLC. Preferably, a chiral α1-acid glycoprotein column (α1-AGP column), containing 183 mg α1-AGP/g solid phase. The enantiomers were resolved using a mobile phase composition of phosphate buffer pH7.0 with addition of 2% v/v of ethanol (95% v/v) and 1.95 mM N,N-dimethyloctylamine.

Cytotoxicity and Efficacy

Cytotoxicity is evaluated by measuring cell survival after exposure to the relevant pharmacologically active agent. Cytotoxicity for purposes of mucosal delivery typically is determined by the level of tetrazolium salt reduction accomplished by surviving cells, preferably over four orders of magnitude. If the level of tetrazolium salt reduction is decreased, then cytotoxicity exists. One assay for measuring tetrazolium salt reduction is the WST-1 assay (Boehringer Mannheim) using L929 lung fibroblast cells. Other known assays include, but are not necessarily limited to assays which measure lactose dehydrogenase (“LDH”), which is released by cells upon death, and/or assays which measure the rate of DNA synthesis.

Various assays also exist for identifying a highest pharmacological activity enantiomer of a given pharmacologically active agent. Where the pharmaceutical activity is as a histamine antagonist, IL-6 production by HUVEC cells is a cell biomarker of histamine activity and is used to assess the relative antagonistic activity of prospective H₁ blockers. IL-6 production in human endothelial cells is known to be induced by histamine due to H₁ and H₂ receptor binding with H₁ the dominant effect. Delneste, et al. As H₁ antagonism is directly linked to reduced emesis during motion sickness treatment, this assay serves as an in vitro methodology for the selection of potential motion sickness and antihistamine candidates. Realtime RT-PCR analysis of IL-6 mRNA synthesis in HUVEC cells stimulated with histamine is employed as an in vitro assay for the analysis of the relative efficacy of potential antihistaminic agents.

A preferred assay for measuring activity of phenothiazine and other histamine antagonists comprises: providing at least a first viable culture and a second viable culture comprising Huvec cells; exposing the first viable culture to a first combination comprising histamine and the (+)-enantiomer of the phenothiazine under conditions effective to inhibit IL-6 mRNA expression; exposing the second viable culture to a combination comprising histamine and the (−)-enantiomer of the phenothiazine under conditions effective to inhibit IL-6 mRNA expression; measuring inhibition of IL-6 mRNA expression induced by the first combination and the second combination after at least four hours to identify a (+)-enantiomer inhibition value and a (−)-enantiomer inhibition value; and selecting as the highest pharmacological activity enantiomer the enantiomer having the greater inhibition value selected from the group consisting of the (+)-enantiomer inhibition value and the (−)-enantiomer inhibition value.

In a preferred embodiment, the method further comprises providing a third viable culture comprising Huvec cells as a control; exposing the third viable culture to a third combination comprising histamine in the absence of the phenothiazine under conditions effective to induce IL-6 mRNA expression; and, measuring IL-6 mRNA expression induced by the third combination after at least four hours to identify a control expression value.

In a preferred embodiment, the method further comprises providing a fourth viable culture comprising Huvec cells; exposing the fourth viable culture to a fourth combination comprising histamine and a racemate mixture of the phenothiazine under conditions effective to inhibit IL-6 mRNA expression; measuring inhibition of IL-6 mRNA expression induced by the fourth combination after at least four hours to identify a racemate inhibition value. Depending on the results, this embodiment may comprise identifying the racemate mixture of the phenothiazine as the highest activity candidate.

The data for each enantiomer is compared to that of the racemate and the other enantiomers of the experimental group and a ‘highest efficacy’ (HE) candidate is selected. This data is a significant indicator for efficacy against motion sickness and/or allergy.

Formulation of Pharmaceutical Preparation

Pharmaceutical preparations comprising microcapsules, as described herein, are useful to deliver substantially any pharmacologically active agent selected from the group consisting of antihistamines and anticholinergics across the blood-brain barrier.

If one enantiomer of the particular pharmacologically active agent is superior, then the most potent and/or less cytotoxic enantiomer is used alone. In a preferred embodiment, the pharmacologically active agent is a phenothiazine, most preferably a single, most active enantiomer of the phenothiazine. In a preferred embodiment, the phenothiazine is selected from the group consisting of the (+)-enantiomer of promethazine and the (−)-enantiomer of ethopropazine.

The microcapsules are fabricated by the disk process. D. C. Johnson et al. J. Gas Chrom, 3, 345-347, (1965), incorporated herein by reference. The microcapsules comprise a core and a shell.

The core of the microcapsule preferably comprises an excipient. Suitable excipients include, but are not necessarily limited to mono-, di-, and triglycerides. Suitable mono- and/or di-glycerides are selected from the group consisting of MYVEROL™ and MYVOCET™ which are commercially available from Gillco Ingredients. Suitable triglycerides are selected from the group consisting of stearate, hydrogenated palm oil, cottonseed oil, soybean oil, and combinations thereof. The hydrogenated palm oil preferably is partially hydrogenated palm oil, most preferably STEARINE-27, a partially hydrogenated palm oil with a melting point of −135° F. STEARINE-27 is commercially available from Loders-Croklaan. In one embodiment, the triglyceride is mixed with the pharmacologically active agent. The core of the microcapsule also may comprise one or more absorption enhancer(s).

The microcapsules preferably are over coated with a release retardant. Suitable release retardants or shell materials include, but are not necessarily limited to shellac and ethylcellulose, most preferably ethylcellulose of premium grade from about 4 to about 10, preferably comprising an ethoxyl content of from about 45 wt. % to about 47 wt. %. In a preferred embodiment, a 5% solution of ethylcellulose in 80% toluene and 20% ethanol has a viscosity of from about 9 cP to about 11 cP at 25° C. The release retardant is effective to slow the release of the pharmacologically active agent and to reduce, and preferably to prevent mucosal tissue irritation, preferably nasal tissue irritation. The shell of the microcapsules also may comprise one or more absorption enhancer(s).

In a preferred embodiment, the pharmaceutical preparation comprises microcapsules in combination with one or more absorption enhancer(s). The one or more absorption enhancer(s) may be incorporated into the microcapsules themselves, or the absorption enhancer(s) may be incorporated into a carrier gel or cream. In a preferred embodiment, the absorption enhancer(s) are incorporated into the carrier gel or cream. The absorption enhancer(s) preferably are effective to transport the pharmacologically active agent through mucosal epithelial cells at a mucosal transport rate that is substantially the same as the controlled release rate from the microcapsules. Suitable absorption enhancers include, but are not necessarily limited to those selected from the group consisting of glycodeoxycholate (GDC), dimethyl-cyclodextrin, L-α-lysophosphatidylcholine (LPC), polyethylene glycol (PEG), glycofurol, and mixtures thereof. I. Gill, A. N. Fisher, M. Hinchcliffe, J. Whetstone, R. DePonte, L. Illum, “Cyclodextrins as Protection Agents Against Enhancer Damage in Nasal Delivery Systems”, Eur. J. Pharm. Sci., 1 (5), 1994, pg. 235-248, incorporated herein by reference. A preferred absorption enhancer PEG/glycofurol, more preferably 30/70 wt./wt. PEG/glycofurol, most preferably 30/70 wt./wt. PEG 400/glycofurol.

In a preferred embodiment, the pharmaceutical preparation comprises a microcapsule- gel or cream formulation comprising a suitable carrier. Suitable carriers include, but are not necessarily limited to polyethylene glycol (PEG), glycofurol, laureth-5, 6 or 9, aquaphor, plurfect, poloaxamer, and mixtures thereof, and the like. A preferred PEG is PEG 400. In a preferred embodiment, suitable for nasal delivery, a carrier gel or cream that will not irritate the nasal tissue or inhibit the ciliary beat frequency of the nostril is used.

Preferred pharmacologically effective formulations comprise microcapsules comprising the pharmacologically active agent and an absorption enhancer selected from the group consisting of glycodeoxycholate (GDC), L-α-lysophosphatidylcholine (LPC), and mixtures thereof. In a most preferred embodiment, the pharmacologically effective formulation further comprises a carrier comprising a gel or cream that does not irritate the nasal tissue or inhibit the ciliary beat frequency of the nostril. Preferred carriers are selected from the group consisting of polyethylene glycol, glycofurol, laureth-5, 6 or 9, aquaphor, plurfect, poloaxamer, and mixtures thereof.

Method of Delivery

The pharmaceutical preparation may be delivered in a variety of ways. In a preferred method, the pharmaceutical formulation comprising a pharmacologically active agent is mucosally delivered. In a preferred embodiment, the mucosal delivery is nasal delivery.

The method is effective to enable delivery of the pharmacologically active agent across the blood brain barrier. In a most preferred embodiment, in which the pharmacologically active agent is nasally administered, the microcapsules also can deliver the pharmacologically active agent through the axonal nerve found in the ostium, bypassing the blood brain barrier.

The following examples will better illustrate the application:

### EXAMPLE 1

### Resolution of Enantiomers

Enantiomers of PMZ were prepared, purified, and characterized. A chiral-high performance liquid chromatographic (HPLC) method was developed to enable analysis of the optical purity of the enantiomers prepared.

The methods developed for making the PMZ enantiomers are described below:

Promethazine Base Conversion:

1. To promethazine hydrochloride (12.5 g, 0.039 mol), obtained from Sigma (lot #128H1474) added 100 mL ether and 25 mL 2M sodium hydroxide (0.045 mol). The resulting suspension was shaken and the ether layer was collected. The aqueous layer was extracted twice with ether. The combined ether layers were dried over magnesium sulfate. Rotary evaporation gave 10 g (0.035 mol) promethazine. Yield 90%.

Promethazine-D-tartrate:

2. Promethazine (10 g, 0.035 mol) dissolved in 80 mL acetone was heated in a 60° C. bath while dibenzoyl-D-tartaric acid (12.789 g, 0.036 mol) was added. The resulting clear yellow solution was left at ambient temperature for 3 days.

3. A heavy precipitate formed which was filtered off and recrystallized from ethanol four times to give 4.0 g promethazine dibenzoyl-D-tartrate white crystals.

4. Promethazine-D-tartrate was converted to promethazine by reaction with sodium hydroxide aqueous solution in ether. Ether layer was separated. The aqueous layer was extracted with ether and the combined ether layer was dried over magnesium sulfate. Rotary evaporation gave 1.6 g promethazine.

5. (−)-Promethazine hydrochloride was obtained by precipitation of promethazine with 2M HCl/ether. After vacuum drying 1.34 g off-white powder was obtained.

Promethazine-L-tartrate:

6. From the acetone mother liquor (Step 2) 11.3 g of brownish liquid was obtained after rotary evaporation. This liquid was converted to promethazine 3.6 g (similar to Step 4).

7. To 3.6 g promethazine obtained from the previous step, 36 mL acetone was added, heated in a 60° C. bath and 4.6040 g dibenzoyl-L-tartaric acid was added. The resulting clear solution was left at ambient for 3 days.

8. A heavy precipitate formed which was filtered off and recrystallized (from ethanol 3 times, once from acetone, once more from ethanol) to give 1.2 g promethazine dibenzoyl-L-tartrate white crystals.

9. Similar to Step 4, promethazine-L-tartrate was converted to promethazine.

10. (+)-Promethazine hydrochloride was obtained by precipitation of promethazine with 2M HCl/ether. After vacuum drying, 0.48 g of off-white powder was obtained (purity 99.87% by HPLC).

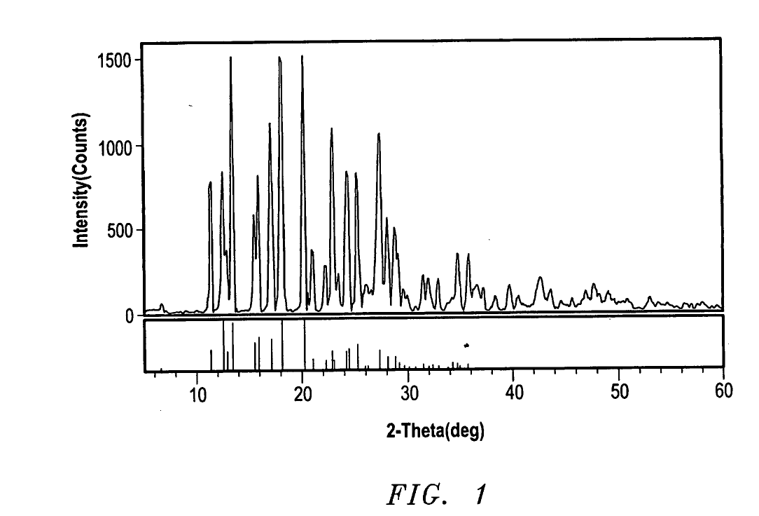

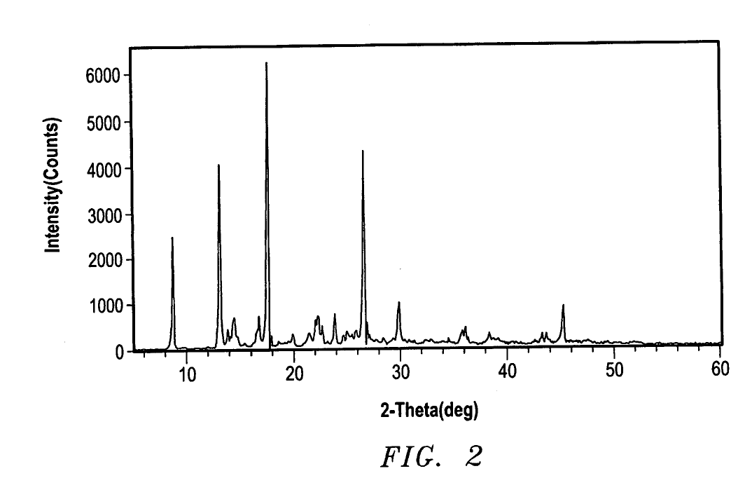

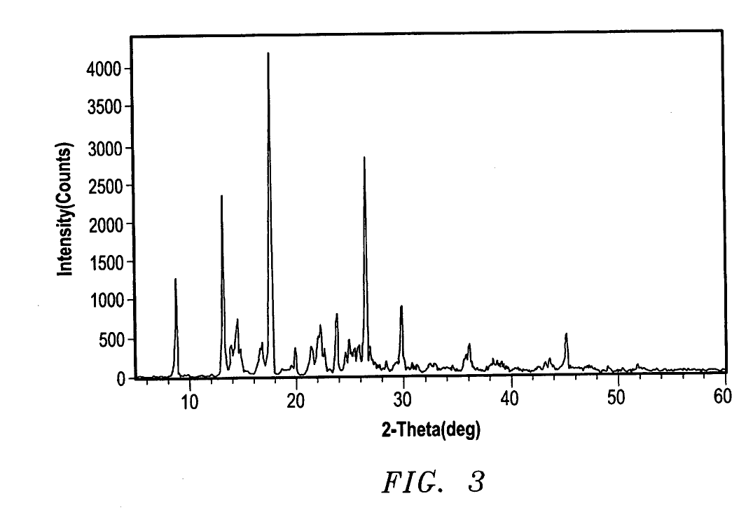

Repeating Steps 1-5 with 5.7703 g promethazine gave about 0.95 g (−)-promethazine hydrochloride as an off-white powder (purity 99.82% by HPLC). X-ray of the promethazine racemate and enantiomers has also been completed and shows that the pure enantiomers are different crystal forms than the racemate (FIGS. 1, 2, and 3). Optical rotation was measured at 27° C. in water.

### EXAMPLE 2

### In Vitro Cytotoxicity of Enantiomers

Enantiomer cytotoxicity was evaluated by measuring cell survival using the WST-1 assay (Boehringer Mannheim). Cells, L929 lung fibroblast, were grown in culture until confluent. The cells were then treated with the enantiomer dissolved in DMSO (dimethyl sulfoxide, 1 g %) for 1 and 18 hours. Enantiomer cytotoxicity was tested over a four-fold range of concentration.

Following enantiomer incubation, the conversion of WST1 reagent by cells was measured spectrophotometrically as an indicator of cell number and, hence, cell survival. A total of 8 replicate wells of each test concentration were used per assay. One factor analysis of variance (ANOVA), using Fisher's LSD test for post-hoc analysis, was used to determine if the effects of the test substances were significant at the p<0.05 level for each concentration tested versus non-treated controls and DMSO-only treated controls. The data indicates that Ethopropazine, Trimeprazine and Promethazine are all cytotoxic at concentrations greater than 10⁻⁵ M.

### EXAMPLE 3

### Promethazine Enantiomer Inhibition of Histamine Activity

Huvec cells were plated and grown to confluence in 6-well plates. At confluence, the cells were treated with either Histamine (10⁻⁴ M, H), Promethazine racemate (10⁻⁵ M) and Histamine (10⁻⁴ M, R), Promethazine (+) enantiomer (10⁻⁵ M) and Histamine (10⁻⁴ M), Promethazine (−) enantiomer (10⁻⁵ M) and Histamine (10⁻⁴ M) or left untreated (U/T) for 5 hours. Total RNA was isolated using Tri-reagent and subjected to reverse transcription polymerase chain reaction (RT-PCR) analysis of IL-6 production using semiquantitative analysis against HPRT expression (control gene).

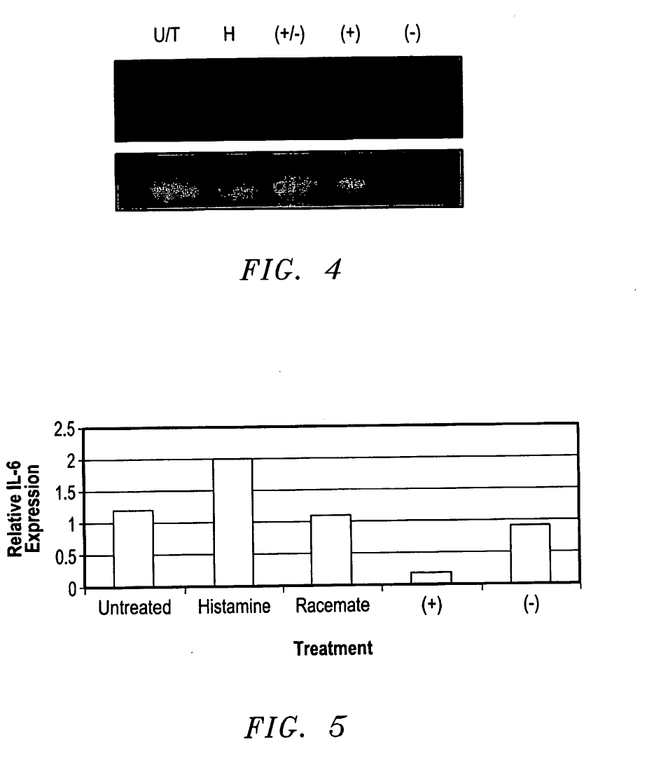

As shown in FIG. 4 and FIG. 5, IL-6 was produced by the cells endogenously. Histamine alone stimulated a 50% increase in IL-6 mRNA production. As plotted in FIG. 5, Promethazine racemate inhibited histamine stimulation of IL-6 production by 50% of that of cells stimulated only with histamine. The (+) enantiomer reduced IL-6 production to 90% of the histamine stimulated cell while the (−) enantiomer produced a 50% reduction in IL-6 production or that approximately equal to that of the control cells. This data demonstrates the major antihistamine activity associated with the Promethazine moiety resides in the (+) enantiomer.

### EXAMPLE 4

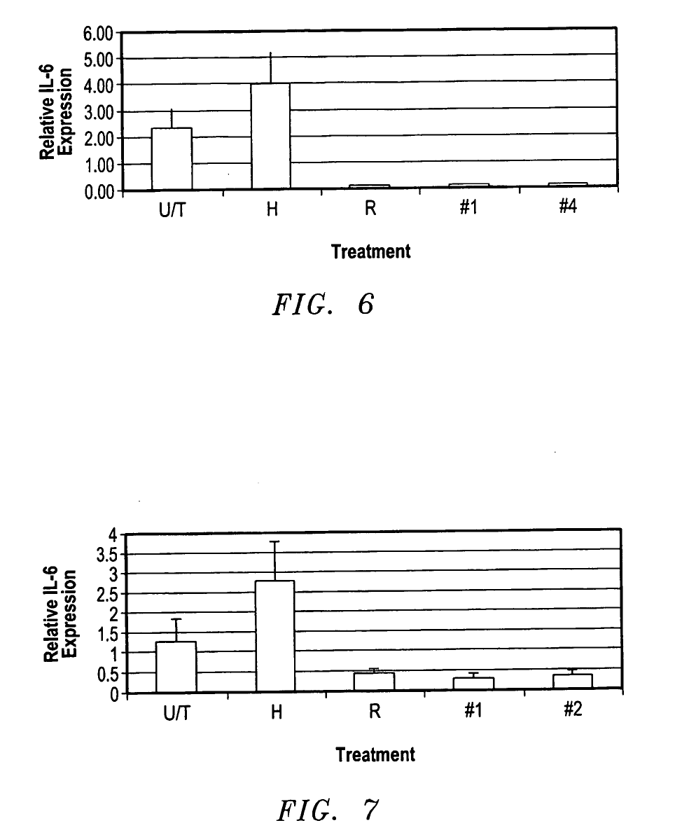

Ethopropazine (EPZ), obtained from Sigma as the racemate ethopropazine hydrochloride, was resolved using the procedures in Example 1 and subjected to the assays described in Examples 2 and 3. The results are given in FIG. 6.

### EXAMPLE 6

Trimeprazine (TPZ), obtained from Sigma as the racemate, was resolved by preparative column chromatography using CHIRALCEL® OJ-H® preparative column eluting with 99.9% methanol/0.1% diethylamine at room temperature. The isolated enantiomers were subjected to the assays described in Examples 2 and 3. The results are given in FIG. 7.

### EXAMPLE 7

Racemic ethopropazine hydrochloride salt was mixed with methylene chloride and 2M sodium hydroxide. The resulting suspension was agitated and organic layer collected. After drying the solvent was removed by rotary evaporation to give racemic ethopropazine base (4.0 g, 0.013 mol) that reacted with dibenzoyl-D-tartaric acid (4.4 g, 0.012 mol) in acetone with agitation. A white precipitate was collected after a few hours. After two recrystallization steps from absolute ethanol, a 99+% crystal was obtained which was converted to ethopropazine hydrochloride salt. Yield: 20%. From the mother liquor, another diastereomeric salt was obtained as white precipitate, which was also recrystallized twice from absolute ethanol before converting to hydrochloride salt. Yield: 20%.

The following were the peak results from chiral HPLC chromatograms of the ethopropazine HCl racemate:

| | Name | RT | Area | Height |
|---|---|---|---|---|
| 1 | (−)-EPZ | 5.334 | 1146985 | 83789 |
| 2 | (+)-EPZ | 5.912 | 1177960 | 72702 |

The following were the peak results from chiral HPLC chromatograms of one of the recrystallized salts, which was determined by HNMR to be the (−)-enantiomer of ethopropazine HCl:

| | Name | RT | Area | Height |
|---|---|---|---|---|
| 1 | (−)-EPZ | 5.348 | 1305178 | 94629 |

The following were the peak results from chiral HPLC chromatograms of the other recrystallized salt, which was determined by HNMR to be the (+)-enantiomer of ethopropazine HCl:

| | Name | RT | Area | Height |
|---|---|---|---|---|
| 2 | (+)-EPZ | 5.893 | 2521326 | 157674 |

Optical rotations were measured in water at 27° C.

### EXAMPLE 8

### Microencapsulation Technology for Phenothiazines

A hot melt of STEARINE-27 (Loders-Croklaan) with PMZ (Sigma) loading of 40% was used to make the core microcapsules by running off the disk at 6000 RPM at 50-55° C. Ethocel (10%) solutions in ethylacetate:acetone (60:40 wt/wt) were used to coat the PMZ or the PMZ stearine microcapsules. A picture of the PMZ microcapsules is displayed in FIG. 8.

### EXAMPLE 9

### In Vitro Cytotoxicity of Enantiomers

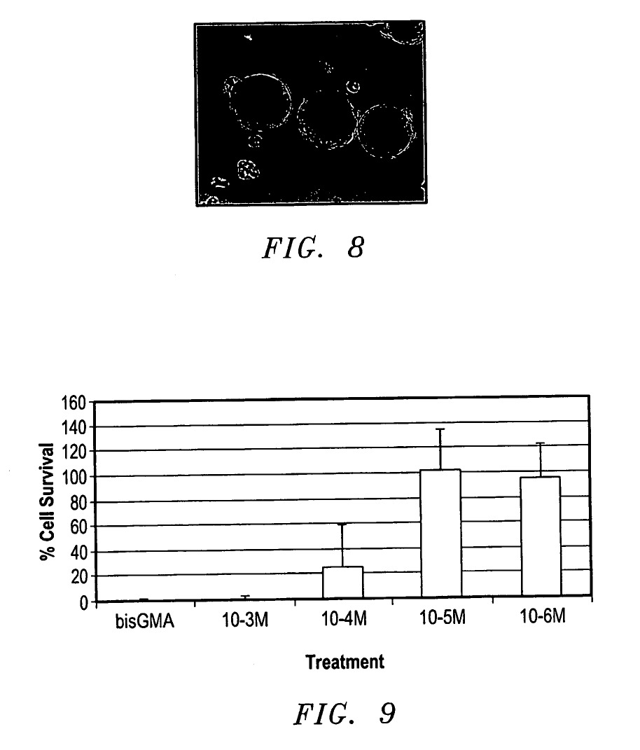

Enantiomer cytotoxicity was evaluated by measuring cell survival using the WST-1 assay (Boehringer Mannheim). Cells, L929 lung fibroblast, were grown in culture until confluent. The cells were then treated with the enantiomer dissolved in DMSO (dimethyl sulfoxide, 1 g %) for 1 and 18 hours. Enantiomer cytotoxicity was tested over a fourfold range of concentration. Following enantiomer incubation, the conversion of WST1 reagent by cells was measured spectrophotometrically as an indicator of cell number and, hence, cell survival. Eight replicate wells of each test concentration were used per assay. One factor analysis of variance (ANOVA) using Fisher's LSD test for post-hoc analysis, was used to determine if the effects of the test substances were significant at the p<0.05 level for each concentration tested versus nontreated controls and DMSO-only treated controls. The data is plotted in FIGS. 9, 10, and 11. BisGMA was used as the control since it has been shown to be significantly cytotoxic in this assay by previous studies.

The data indicates that the PMZ and enantiomers are cytotoxic at concentrations of 10⁻⁴ M and greater. EPZ and TPZ were also found to be cytotoxic at 10 ⁻⁴ M and greater.

### EXAMPLE 10

### In Vivo Analysis of Nasal Irritation and PMZ Uptake

This study was undertaken to evaluate the effect of various formulations of promethazine (PMZ) on the rat nasal mucosa when given via a topical nasal mechanism. Six formulations were evaluated:
1) Saline alone (negative control);
2) Promethazine HCl in Saline (positive control);
3) Promethazine HCl in PBS (ph 7.2);
4) Promethazine Freebase in a PEG 400-Glycofurol (30/70 wt./wt.) carrier
5) Encapsulated Promethazine (as above) in a PEG 400-Glycofurol (30/70 wt./wt.) carrier;
6) The PEG 400-Glycofurol (30/70 wt./wt.) carrier alone.
   Sprague-Dawley rats (200 g) were obtained from Harlan and acclimated to housing at a laboratory animal facility for 1 week prior to experimentation. On the day of the experiment, the animals were separated at random into six groups of eight animals each. Each animal was anesthetized with ketamine/xylazine/acetylpromazine, premixed as a cocktail (44.0/8.4/1.0 mg/kg body weight, 0.15 cc of cocktail per 100-g body weight) and following compete sedation, a 5-μL aliquot of PMZ formulation (125 mg/mL) was placed in the left nostril with a micropipette (note: for the encapsulated formulation, 15 μL of formulation was administered as the PMZ concentration was only 42 mg/mL). At 30 minutes post-formulation application, a 0.5-mL aliquot of blood was drawn from the infraorbital sinus into heparinized vials and the animals returned to their cages.

At 24 hours each animal was again given an aliquot of anesthesia cocktail as above. The abdominal segment of the aorta was then exposed and 1 ml of blood was drawn into heparinized tubes. The animal was decapitated, the anterior integument and lower jaw removed and a 10-mL volume of Millongs solution (5% Formalin in PBS) gently injected into the nasal cavity. The upper head was then fixed in 50 mL of Millongs for 48 hours with one fixative change. The head was decalcified in buffered formic acid for 14 days with changes of solution every 2 days until no evidence of calcium was found. At that time, the specimen was dissected into four segments stretching from the anterior to posterior nasal cavity (numbered C1 through C4) and paraffin embedded using standard protocols. Sections (5 microns) were cut from each tissue specimen and stained with H&E. The histology was documented with an Olympus microscope using Image Pro software. Blood samples were centrifuged at 1000×g for 5 minutes to pellet the cells and the plasma removed to pre-labeled vials which were stored at −80° C.

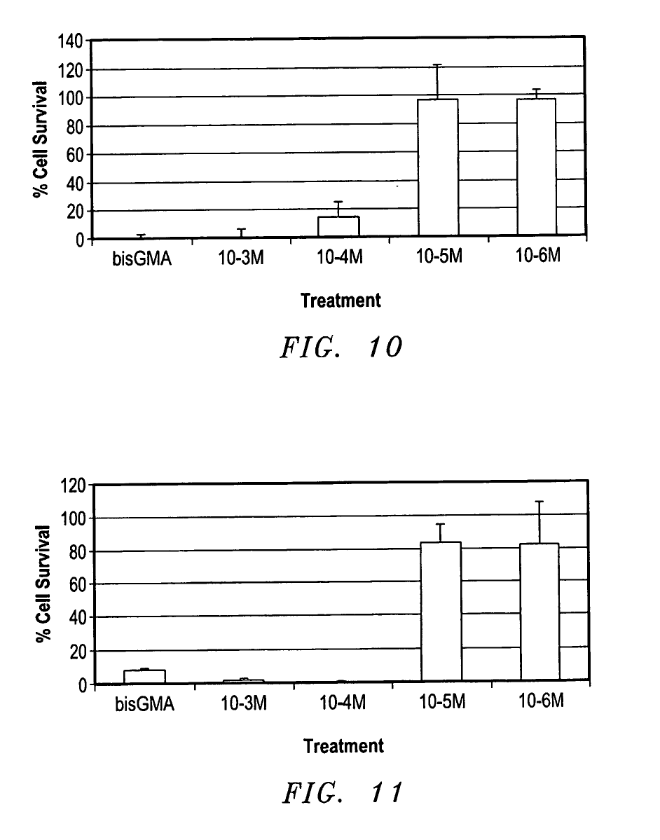

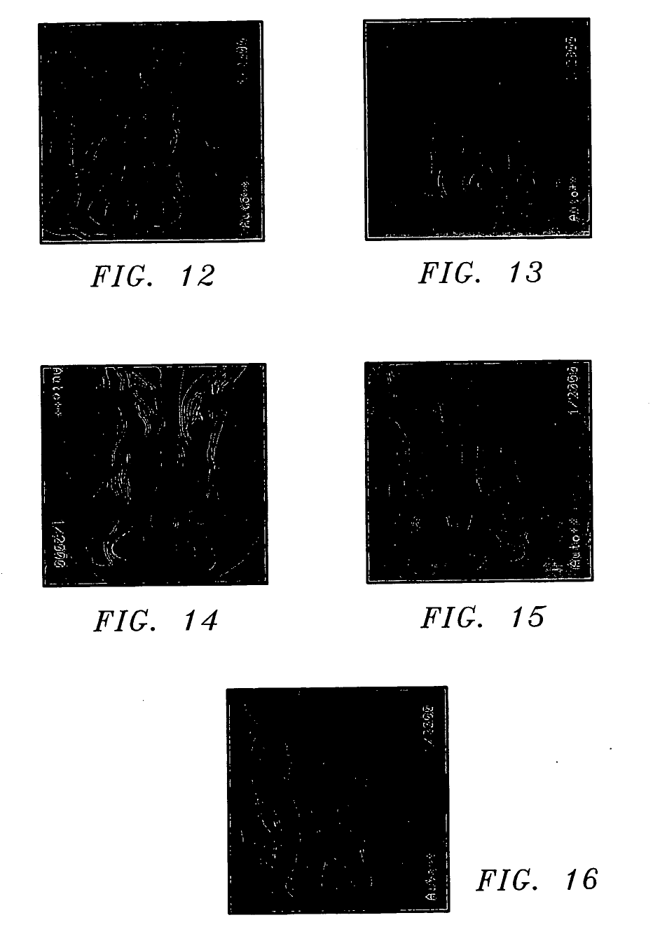

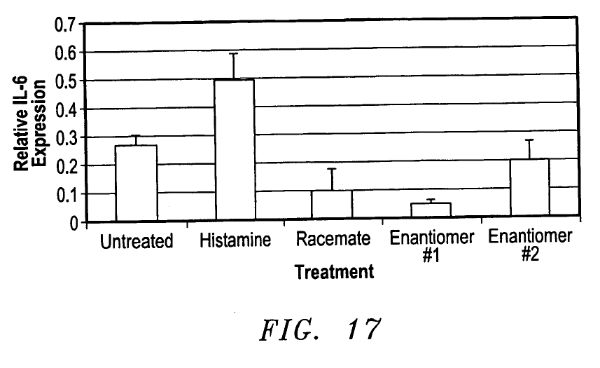

The results of the experiment are shown in Table 1, below, and the histology is shown in the FIGS. 12-16. Saline administered alone produced no effect (FIG. 12). The PMZ-Saline formulation produced a significant inflammatory response in the ventral aspect of the anterior segment of the nasal epithelium (FIG. 13). The PMZ-PBS formulation produced effects comparable to that seen with the PMZ-Saline formulation alone, extensive inflammation of the ventral aspect of the anterior segment (FIG. 14) was evident in all animals tested. As with PMZ-Saline, the inflammation was restricted to the dosed segment. The PMZ-Freebase produced a small inflammatory response in only two animals (FIG. 15). The encapsulated PMZ formulation and the PEG 400-Glycofurol carrier did not produce an inflammatory response in any animal (FIG. 16).

TABLE 1
Individual Response to Formulation

| | ANIMAL | | | | | | | |
|---|---|---|---|---|---|---|---|---|
| Treatment | 1 | 2 | 3 | 4 | 5 | 6 | 7 | 8 |
| Saline | − | − | − | − | − | − | − | − |
| PMZ in Saline | + | + | + | + | + | + | + | + |
| PMZ in Buffer | + | + | + | + | + | + | + | + |
| PMZ Freebase | + | + | − | − | − | − | − | − |
| Encapsulated PMZ | − | − | − | − | − | − | − | − |
| PEG 400-Glycofurol | − | − | − | − | − | − | − | − |

Blood PMZ concentration was measured in the 30 minute post-treatment plasma samples (Table 2). PMZ was detected in all animals tested. Microencapsulated PMZ delivery was not statistically different from that of the PMZ in freebase.

TABLE 2
PMZ Concentration (mg/L) in Plasma at
30 Minutes Post Application

| Treatment | PMZ | PMZSO | DMPMZ | DMPMZSO |
|---|---|---|---|---|
| PMZ IN SALINE | 212 | 59.5 | 4.2 | 6.6 |
| PMZ IN BUFFER | 265 | 48.4 | 4.8 | 6.2 |
| PMZ CAPSULE | 173.4 | 47.4 | 4.3 | 4.6 |

### EXAMPLE 11

The procedures of Example 3 were repeated using the (+)-enantiomer (#2) and the (−)-enantiomer (#1) of EPZ at 10⁻⁵ molar and 10⁻⁶ molar, and TPZ at 10⁻⁵ molar. The results are given in FIGS. 6, 7, and 17. The results indicate that the (−)-enantiomer of EPZ was significantly more active than the other enantiomer, or the racemate (FIG. 17). Although TPZ did not demonstrate a highest activity enantiomer at 10⁻⁵ molar (FIG. 7), it is expected that the use of a lower dose of TPZ will resolve which is the more active enantiomer.

Persons of ordinary skill in the art will recognize that many modifications may be made to the present invention without departing from the spirit and scope of the present invention. The embodiment described herein is meant to be illustrative only and should not be taken as limiting the invention, which is defined in the claims.

1. A pharmaceutical preparation adapted for mucosal delivery of a pharmacologically effective dose of a pharmacologically active agent to a mammal, said pharmaceutical preparation comprising microcapsules adapted to provide controlled release of said pharmacologically effective dose, said microcapsules comprising a core and a shell, said shell comprising a release retardant, said core comprising said pharmacologically active agent and an excipient, wherein said pharmacologically active agent is selected from the group consisting of antihistamines and anticholinergics.

2. A pharmaceutical preparation adapted for mucosal delivery of a pharmacologically effective dose of a pharmacologically active agent to a mammal, said pharmaceutical preparation comprising microcapsules adapted to provide controlled release of said pharmacologically effective dose, said microcapsules comprising a shell and a core, said core comprising a quantity of a single enantiomer of said pharmacologically active agent, wherein said pharmacologically active agent is selected from the group consisting of antihistamines and anticholinergics.

3. A pharmaceutical preparation adapted for mucosal delivery of a pharmacologically effective dose of a pharmacologically active agent to a mammal, said pharmaceutical preparation comprising one or more absorption enhancers and microcapsules adapted to provide controlled release of said pharmacologically effective dose of said pharmacologically active agent, wherein said pharmacologically active agent is selected from the group consisting of antihistamines and anticholinergics.

4. A method for mucosal delivery of a pharmacologically effective dose of a pharmacologically active agent to a mammal comprising:
providing a pharmaceutical preparation comprising microcapsules comprising a core and a shell, said shell comprising a release retardant, said core comprising a pharmacologically active agent and an excipient, wherein said pharmacologically active agent is selected from the group consisting of antihistamines and anticholinergics; and,
mucosally administering said pharmaceutical preparation to said mammal.

5. A method for mucosal delivery of a pharmacologically effective dose of a pharmacologically active agent to a mammal comprising:
providing a pharmaceutical preparation comprising microcapsules adapted to provide controlled release of said pharmacologically effective dose, said microcapsules comprising a shell and a core, said core comprising a quantity of a single enantiomer of said pharmacologically active agent, wherein said pharmacologically active agent is selected from the group consisting of antihistamines and anticholinergics; and
mucosally administering said pharmaceutical preparation to said mammal.

6. A method for mucosal delivery of a pharmacologically effective dose of a pharmacologically active agent to a mammal comprising:
providing a pharmaceutical preparation comprising one or more absorption enhancers and microcapsules adapted to provide controlled release of said pharmacologically effective dose of said pharmacologically active agent, wherein said pharmacologically active agent is selected from the group consisting of antihistamines and anticholinergics.
mucosally administering said pharmaceutical preparation to said mammal.

7. A pharmaceutical preparation for mucosal delivery of a pharmacologically active agent to a mammal without cytotoxicity to mucosal epithelial cells, said pharmaceutical preparation comprising:
microcapsules comprising a shell and a core comprising a quantity of one or more pharmacologically active agents selected from the group consisting of antihistamines and anticholinergics, said microcapsules being adapted to release said one or more pharmacologically active agents at a release rate, wherein cytoxicity is predicted due to a factor selected from the group consisting of said release rate and inherent cytotoxicity of said pharmacologically active agent; and
one or more absorption enhancers effective to produce a mucosal transport rate which is substantially the same as said release rate of said pharmacologically active agent, thereby preventing said cytotoxicity.

8. A method for mucosal delivery of a pharmacologically active agent to a mammal, said method comprising:
providing a pharmaceutical preparation comprising microcapsules comprising a shell and a core, said core comprising one or more pharmacologically active agents selected from the group consisting of antihistamines and anticholinergics, said microcapsules being adapted to provide a release rate of said pharmacologically active agent, wherein cytotoxicity is predicted due to a factor selected from the group consisting of said release rate and inherent cytotoxicity of said pharmacologically active agent; and,
mucosally delivering said pharmaceutical preparation to a mammal under conditions effective to produce a mucosal transport rate which is substantially the same as said release rate of said pharmacologically active agent, thereby preventing said cytotoxicity.

9. The pharmaceutical preparations and methods of any of claims 1-8 wherein said pharmacologically active agent is phenothiazine.

10. The pharmaceutical preparations and methods of any of claims 1-9 wherein said mucosal delivery comprises nasal mucosal delivery.

11. The pharmaceutical preparations and methods of any of claims 1-10 wherein said pharmaceutical preparations are adapted to avoid cytotoxicity to mucosal epithelial cells upon said mucosal delivery.

12. The pharmaceutical preparations and methods of any of claims 9-11 wherein said phenothiazine has the following general structure: 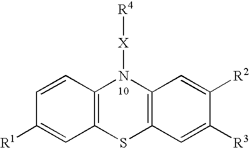
wherein
R¹, R², and R³ have a size substantially equivalent to an alkyl radical having 6 or fewer carbon atoms;
X is selected from the group consisting of a linear or branched alkyl radical and a linear or branched alkenyl group having from about 1 to about 5 carbon atoms;
R⁴ is a tertiary amine or thiol radical having a structure selected from the group consisting of N—(R⁵)₃ and S—R⁵ wherein R⁵ comprises the same or different entities independently selected from the group consisting of hydrogen, alkyl radicals and alkenyl radical having from about 1 to about 4 carbon atoms, cyclic alkylene groups and heterocyclic alkylene groups having from about 4 to about 6 carbon atoms comprising a heterocyclic element selected from the group consisting of nitrogen or sulfur.

13. The pharmaceutical preparations and methods of claim 12 wherein R¹, R², and R³ independently are further selected from the group consisting of ionizable groups selected from the group consisting of ammonium, sulfonium, and phosphonium groups and esters thereof.

14. The pharmaceutical preparations and methods of claim 13 wherein said esters comprise linear or branched alkyl groups comprising from about 1 to about 5 carbon atoms.

15. The pharmaceutical preparations and methods of any of claims 12-14 wherein R¹, R², and R³ independently are selected from the group consisting of hydrogen, a hydroxyl radical, an alkoxy radical comprising a branched or unbranched alkyl radical having a total of from about 1 to about 6 carbon atoms, an acyloxy radical comprising a branched or unbranched alkyl radical having a total of from about 1 to about 6 carbon atoms, a substituted or unsubstituted branched or unbranched alkyl radical having a total of from about 1 to about 6 carbon atoms, a substituted or an unsubstituted phenyl radical or a substituted or an unsubstituted benzyl radical wherein said substituted radicals comprise substituents selected from the group consisting of hydroxyl radicals, halogens, alkyl radicals having a total of from about 1 to about 6 carbon atoms, cyclic alkylene groups and heterocyclic alkylene groups having from about 4 to about 6 carbon atoms comprising a heterocyclic element selected from the group consisting of nitrogen or sulfur.

16. The pharmaceutical preparations and methods of any of claims 12-15 wherein R⁵ is selected from the group consisting of alkyl radicals and alkenyl radical having from about 1 to about 3 carbon atoms.

17. The pharmaceutical preparations and methods of any of claims 9-16 wherein said phenothiazine is selected from the group consisting of promethazine, ethopropazine, propiomazine, and trimeprazine.

18. The pharmaceutical preparations and methods of any of claims 9-17 wherein said phenothiazine is promethazine.

19. The pharmaceutical preparations and methods of claim 18 wherein said promethazine consists essentially of the (+)-enantiomer.

20. The pharmaceutical preparations and methods of any of claims 9-17 wherein said phenothiazine is ethopropazine.

21. The pharmaceutical preparations and methods of claim 20 wherein said ethopropazine consists essentially of the (−)-enantiomer.

22. The pharmaceutical preparations and methods of any of claims 1-21 wherein said pharmacologically active agent comprises one or more pharmaceutically acceptable acid addition salt of said phenothiazine.

23. The pharmaceutical preparations and methods of claim 22 wherein said pharmaceutically acceptable acid addition salts are products of reaction between said pharmacologically active agent and an acid selected from the group consisting of hydrochloric acid, hydrobromic acid, hydroiodic acid, sulfuric acid, phosphoric acid, p-toluenesulfonic, methanesulfonic acid, oxalic acid, p-bromophenylsulfonic acid, carbonic acid, succinic acid, citric acid, benzoic acid, and acetic acid.

24. The pharmaceutical preparations and method of claim 22 wherein said pharmaceutically acceptable acid addition salts of said pharmacologically active agent are selected from the group consisting of sulfates, pyrosulfates, bisulfates, sulfites, bisulfites, phosphates, monohydrogenphosphates, dihydrogenphosphates, metaphosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, hydroxybutyrates, glycollates, tartrates, methanesulfonates, propanesulfonates, naphthalene-1-sulfonates, naphthalene-2-sulfonates, and mandelates.

25. The pharmaceutical preparations and method of claim 22 wherein said pharmaceutically acceptable acid addition salts are products of reaction between said pharmacologically active agent and an acid selected from the group consisting of hydrochloric acid, hydrobromic acid, acetic acid, oxalic acid, maleic acid, and fumaric acid.

26. The pharmaceutical preparations and method of any of claims 1-25 wherein said microcapsules comprise from about 0.1 to about 50% by weight of said pharmacologically active agent.

27. The pharmaceutical preparations and method of any of claims 1-25 wherein said microcapsules comprise about 20% by weight of said pharmacologically active agent.

28. The pharmaceutical preparations and methods of any of claims 1-25 wherein said microcapsules release said pharmacologically active agent into isotonic saline at 37° C. over a period of from about 20 to about 360 minutes.

29. The pharmaceutical preparations and methods of any of claims 1-2, 4-5, and 7-28 comprising one or more absorption enhancers.

30. The pharmaceutical preparations and methods of claims 3, 6, and 29 wherein said microcapsules comprise said absorption enhancer.

31. The pharmaceutical preparations and methods of any of claims 3, 6, and 29-30 wherein said core comprises said absorption enhancer.

32. The pharmaceutical preparations and methods of any of claims 1-21 further comprising one or more materials selected from the group consisting of pharmaceutically acceptable carriers and pharmaceutically acceptable diluents.

33. The pharmaceutical preparations and methods of claim 32 wherein said microcapsules comprise said one or more materials selected from the group consisting of pharmaceutically acceptable carriers and pharmaceutically acceptable diluents.

34. The pharmaceutical preparations and methods of claim 33 wherein said core comprises said one or more materials selected from the group consisting of pharmaceutically acceptable carriers and pharmaceutically acceptable diluents.

35. The pharmaceutical preparations and methods of any of claims 1-34 wherein said one or more absorption enhancers are selected from the group consisting of glycodeoxycholate (GDC), dimethyl-cyclodextrin, L-α-lysophosphatidylcholine (LPC), polyethylene glycol (PEG), glycofurol, and mixtures thereof.

36. The pharmaceutical preparations and methods of any of claims 1-35 wherein said one or more absorption enhancers comprise PEG/glycofurol.

37. The pharmaceutical preparations and methods of claim 36 wherein said PEG/glycofurol is PEG 400/glycofurol.

38. The pharmaceutical preparations and methods of any of claims 36-37 wherein said PEG/glycofurol is 30/70 wt./wt. PEG/glycofurol.

39. The pharmaceutical preparations and methods of any of claims 1-38 comprising one or more glyceride selected from the group consisting of mono-, di-, and triglycerides.

40. The pharmaceutical preparations and methods of claim 39 wherein said microcapsules comprise said one or more glyceride.

41. The pharmaceutical preparations and methods of claim 39 wherein said core comprises said one or more glyceride.

42. The pharmaceutical preparations and methods of any of claims 39-41 wherein said one or more glyceride is selected from the group consisting of MYVEROL™, MYVOCET™, and a combination thereof.

43. The pharmaceutical preparations and methods of any of claims 39-41 wherein said one or more glyceride is selected from the group consisting of stearate, hydrogenated palm oil, cottonseed oil, soybean oil, and combinations thereof.

44. The pharmaceutical preparations and methods of claims 39-41 wherein said one or more glyceride is partially hydrogenated palm oil.

45. The pharmaceutical preparations and methods of claim 44 wherein said one or more glyceride is partially hydrogenated palm oil having a melting point of −135° F.

46. The pharmaceutical preparations and methods of any of claims 1-45 wherein said microcapsules comprise a release retardant effective to reduce the rate of release of said pharmacologically active agent.

47. The pharmaceutical preparations and methods of claim 46 wherein said shell comprises said release retardant.

48. The pharmaceutical preparations and methods of any of claims 46-47 wherein said release retardant is selected from the group consisting of ethylcellulose and shellac.

49. The pharmaceutical preparations and methods of any of claims 46-48 wherein said release retardant is ethylcellulose.

50. The pharmaceutical preparations and methods of claim 49 wherein said ethylcellulose is a premium grade ethylcellulose of from about 4 to about 10.

51. The pharmaceutical preparations and methods of any of claims 49-50 wherein said ethylcellulose has an ethoxyl content of from about 45 wt. % to about 47 wt. %.

52. The pharmaceutical preparation of any of claims 49-51 wherein a 5% solution of said ethylcellulose comprising 80% toluene and 20% ethanol has a viscosity of from about 9 cP to about 11 cP at 25° C.

53. The pharmaceutical preparations and methods of any of claims 1-52 being effective to enable delivery of said pharmacologically active agent across the blood brain barrier.

54. The pharmaceutical preparations and methods of any of claims 1-53 being effective to deliver said pharmacologically active agent through the axonal nerve in the ostium.

55. The pharmaceutical preparations and methods of any of claims 1-54 further comprising a carrier comprising a gel or cream.

56. The pharmaceutical preparations and methods of claim 55 wherein said gel or cream that does not irritate the nasal tissue or inhibit the ciliary beat frequency of the nostril.

57. The pharmaceutical preparations and methods of any of claims 55-56 wherein said carrier is selected from the group consisting of polyethylene glycol (PEG), glycofurol, laureth-5, 6 and 9, aquaphor, plurfect, poloaxamer, and mixtures thereof.

58. The pharmaceutical preparations and method of any of claims 1-57 wherein said one or more pharmacologically active agents comprise one or more antihistamines.

59. The pharmaceutical preparations and method of any of claims 1-57 wherein said one or more pharmacologically active agents comprise one or more anticholinergics.

60. The pharmaceutical preparations and methods of any of claims 1-59 wherein cytotoxicity is predicted using the WST-1 assay.

61. A method for alleviating a condition in a mammal selected from the group consisting of motion sickness, allergy, and a combination thereof, said method comprising administering to the mammal a pharmacologically effective amount of a highest pharmacological activity enantiomer of a phenothiazine.

62. The method of claims 61 wherein said phenothiazine has the following general structure: 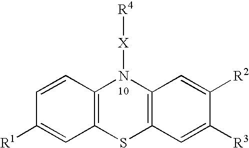
wherein
R¹, R², and R³ have a size substantially equivalent to an alkyl radical having 6 or fewer carbon atoms;
X is selected from the group consisting of a linear or branched alkyl radical and a linear or branched alkenyl group having from about 1 to about 5 carbon atoms;
R⁴ is a tertiary amine or thiol radical having a structure selected from the group consisting of N—(R⁵)₃ and S—R⁵ wherein R⁵ comprises the same or different entities independently selected from the group consisting of hydrogen, alkyl radicals and alkenyl radical having from about 1 to about 4 carbon atoms, cyclic alkylene groups and heterocyclic alkylene groups having from about 4 to about 6 carbon atoms comprising a heterocyclic element selected from the group consisting of nitrogen or sulfur.

63. The method of claim 62 wherein R¹, R², and R³ independently are further selected from the group consisting of ionizable groups selected from the group consisting of ammonium, sulfonium, and phosphonium groups and esters thereof.

64. The method of claim 63 wherein said esters comprise linear or branched alkyl groups comprising from about 1 to about 5 carbon atoms.

65. The method of any of claims 62-64 wherein R¹, R², and R³ independently are selected from the group consisting of hydrogen, a hydroxyl radical, an alkoxy radical comprising a branched or unbranched alkyl radical having a total of from about 1 to about 6 carbon atoms, an acyloxy radical comprising a branched or unbranched alkyl radical having a total of from about 1 to about 6 carbon atoms, a substituted or unsubstituted branched or unbranched alkyl radical having a total of from about 1 to about 6 carbon atoms, a substituted or an unsubstituted phenyl radical or a substituted or an unsubstituted benzyl radical wherein said substituted radicals comprise substituents selected from the group consisting of hydroxyl radicals, halogens, alkyl radicals having a total of from about 1 to about 6 carbon atoms, cyclic alkylene groups and heterocyclic alkylene groups having from about 4 to about 6 carbon atoms comprising a heterocyclic element selected from the group consisting of nitrogen or sulfur.

66. The method of any of claims 62-65 wherein R⁵ is selected from the group consisting of alkyl radicals and alkenyl radical having from about 1 to about 3 carbon atoms.

67. The method of any of claims 61-66 wherein said phenothiazine is selected from the group consisting of promethazine, ethopropazine, propiomazine, and trimeprazine.

68. The method of any of claims 61-67 wherein said phenothiazine comprises promethazine.

69. The method of claim 68 wherein said promethazine consists essentially of the (+)-enantiomer.

70. The method of any of claims 61-67 wherein said phenothiazine comprises ethopropazine.

71. The method of claim 70 wherein said ethopropazine consists essentially of the (−)-enantiomer.

72. The method of any of claims 61-71 wherein said phenothiazine comprises one or more pharmaceutically acceptable acid addition salts of said phenothiazine.

73. The method of claim 72 wherein said pharmaceutically acceptable acid addition salts are products of reaction between said phenothiazine and an acid selected from the group consisting of hydrochloric acid, hydrobromic acid, hydroiodic acid, sulfuric acid, phosphoric acid, p-toluenesulfonic, methanesulfonic acid, oxalic acid, p-bromophenylsulfonic acid, carbonic acid, succinic acid, citric acid, benzoic acid, and acetic acid.

74. The method of claim 72 wherein said pharmaceutically acceptable acid addition salts of said phenothiazine are selected from the group consisting of sulfates, pyrosulfates, bisulfates, sulfites, bisulfites, phosphates, monohydrogenphosphates, dihydrogenphosphates, metaphosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, hydroxybutyrates, glycollates, tartrates, methanesulfonates, propanesulfonates, naphthalene-1-sulfonates, naphthalene-2-sulfonates, and mandelates.

75. The method of claim 72 wherein said pharmaceutically acceptable acid addition salts are products of reaction between said phenothiazine and an acid selected from the group consisting of hydrochloric acid, hydrobromic acid, acetic acid, oxalic acid, maleic acid, and fumaric acid.

76. The method of any of claims 1-75 wherein said single enantiomer or said highest pharmacological activity enantiomer is isolated by a method comprising:
purifying a racemic phenothiazine free base;
mixing said racemic phenothiazine solution and an optically active organic acid under mixing conditions effective to producing a precipitate comprising crystals comprising diasteriomers comprising a reaction product between said optically active organic acid and a corresponding enantiomer of said phenothiazine;
collecting and recrystallizing said precipitate;
converting said precipitate to said corresponding enantiomer of said phenothiazine.

77. The method of claim 76 wherein said purifying a racemic phenothiazine free base comprises
converting a racemic phenothiazine salt to a racemic phenothiazine free base;
dissolving said racemic phenothiazine free base in a volatile organic solvent, producing a phenothiazine free base solution.

78. The method of any of claims 77 wherein said purifying a racemic phenothiazine free base further comprises evaporating said volatile organic solvent.

79. The method of claim 76 wherein said volatile organic solvent is methylene chloride.

80. The method of any of claims 76-79 wherein said mixing conditions comprise acetone as a precipitating solvent.

81. The method of any of claims 76-80 wherein said recrystallizing occurs in the presence of a crystallizing solvent comprising ethanol.

82. The method of any of claims 76-81 wherein said optically active organic acid is optically active dibenzoyl tartaric acid.

83. The method of any of claims 76-82 wherein said phenothiazine is ethopropazine.

84. The method of any of claims 2, 5, and 9-83 wherein said single enantiomer or said highest pharmacological activity enantiomer is identified by a method comprising:
providing at least a first viable culture and a second viable culture comprising Huvec cells;
exposing said first viable culture to a first combination comprising histamine and a composition consisting essentially of (+)-enantiomer of said phenothiazine under conditions effective to inhibit IL-6 mRNA expression;
exposing said second viable culture to a combination comprising histamine and a composition consisting essentially of (−)-enantiomer of said phenothiazine under conditions effective to inhibit IL-6 mRNA expression; and
measuring inhibition of IL-6 mRNA expression by said first combination after at least four hours to identify a (+)-enantiomer inhibition value;
measuring inhibition of IL-6 mRNA expression by said second combination after at least four hours to identify a (−)-enantiomer inhibition value; and
selecting as said highest pharmacological activity enantiomer the enantiomer having the greater inhibition value selected from the group consisting of said (+)-enantiomer inhibition value and said (−)-enantiomer inhibition value.

85. The method of claim 84 further comprising providing a third viable culture comprising Huvec cells as a control;
exposing said third viable culture to a third combination comprising histamine in the absence of said phenothiazine under conditions effective to induce IL-6 mRNA expression;
measuring IL-6 mRNA expression induced by said third combination after at least four hours to identify a control expression value.

86. The method of any of claims 84-85 further comprising providing a fourth viable culture comprising Huvec cells;
exposing said fourth viable culture to a fourth combination comprising histamine and said racemate mixture of said phenothiazine under conditions effective to inhibit IL-6 mRNA expression;
measuring inhibition of IL-6 mRNA expression induced by said fourth combination after at least four hours to identify a racemate inhibition value.

87. The method of claim 86 further comprising identifying said racemate mixture of said phenothiazine as having highest activity when said racemate inhibition value is higher than either said (+)-enantiomer inhibition value and said (−)-enantiomer inhibition value.

88. The method of any of claims 86-87 wherein said measuring said IL-6 mRNA expression comprises:
isolating total RNA in each culture;
subjecting said total RNA in each culture to reverse transcription polymerase chain reaction (RT-PCR) analysis of IL-6 production using semiquantitative analysis against HPRT expression (control gene).

89. A method for resolving (+) enantiomer and (−) enantiomer of ethopropazine, said method comprising:
purifying a racemic ethopropazine free base;
mixing said racemic ethopropazine free base solution and an optically active organic acid under mixing conditions effective to produce a precipitate comprising crystals comprising diasteriomers comprising a reaction product between said optically active organic acid and a corresponding enantiomer of said ethopropathiazine; and,
recrystallizing at least one of said diasteriomers.

90. The method of claim 89 wherein said purifying a racemic ethopropazine free base comprises converting a racemic ethopropazine salt to a racemic ethopropazine free base by dissolving said racemic ethopropazine salt in a volatile organic solvent in contact with a aqueous solution of sodium hydroxide, thereby producing an ethopropazine free base solution.

91. The method of claim 90 wherein said aqueous solution of sodium hydroxide is 2M.

92. The method of any of claims 88-91 wherein said purifying a racemic ethopropazine free base further comprises evaporating said volatile organic solvent.

93. The method of any of claims 88-92 wherein said volatile organic solvent is methylene chloride.

94. The method of any of claims 88-93 wherein said mixing conditions comprise acetone as a precipitating solvent.

95. The method of any of claims 88-94 wherein said recrystallizing occurs in the presence of a crystallizing solvent comprising ethanol.

96. The method of any of claims 88-95 wherein said optically active organic acid is optically active dibenzoyl tartaric acid.

97. The method of any of claims 83-96 further comprising separately recrystallizing both of said diasteriomers.
